(19) United States
(12) Patent Application Publication
(10) Pub. No.: US 2026/0159461 A1
(43) Pub. Date: Jun. 11, 2026

(54) INTEGRATED LIQUID CRACKING PROCESS WITH REVERSE ISOMERIZATION OF ISO-ALKANE TO NORMAL-ALKANE FOR ENHANCED OLEFIN YIELD

(71) Applicant: SABIC Global Technologies B.V., Bergen op Zoom (NL)

(72) Inventors: Zeeshan NAWAZ, Bangalore (IN); Ahmed Suliman AL-ZENAIDI, Bangalore (IN); Pradyut SAMANTA, Bangalore (IN); Thamire Srinavasa Rao, Bangalore (IN)

(73) Assignee: SABIC Global Technologies B.V., Bergen op Zoom (NL)

(21) Appl. No.: 18/707,570

(22) PCT Filed: Dec. 14, 2022
(86) PCT No.: PCT/EP2022/085959

§ 371 Date: May 3, 2024

Related U.S. Application Data
(60) Provisional application No. 63/291,943, filed on Dec. 20, 2021.

(51) Int. Cl. C07C 5/27; C07C 4/02; C07C 7/00; C07C 7/04; C10G 45/02; C10G 45/62; C10G 67/02

(52) U.S. Cl. CPC C07C 5/2724; C07C 4/02; C07C 7/005; C07C 7/04; C10G 45/02; C10G 45/62; C10G 67/02; C07C 2523/42; C10G 2300/1025; C10G 2400/20

(57) ABSTRACT

Integrated systems and methods of processing natural gas liquid (rich in C₅ content) to produce one or more light olefins are disclosed. The systems include a reverse isomerization reactor for reverse isomerizing iso-alkane to normal-alkane. The disclosed integrated system includes the reverse isomerization of iso-pentane to normal-pentane before cracking to form one or more light olefins.

### CROSS REFERENCE TO RELATED APPLICATIONS

None.

### FIELD OF INVENTION

The present disclosure generally relates to an integrated method of processing natural gas liquid (NGL). More specifically, the present disclosure relates to a method of processing natural gas liquid that involves reverse isomerizing iso-alkane to normal-alkane to increase the yield of olefins in a subsequent cracking operation.

### BACKGROUND OF THE INVENTION

Currently, in the chemical industry, as well as other industries, there is increased focus on minimizing carbon loss from, and maximizing productivity and energy efficiency of, production processes. Natural gas liquids are one of the significant sources/feedstocks for petrochemicals. The largest component of natural gas liquids is pentane. But there are other heavier hydrocarbon components that may be present in the natural gas liquids, such as hexane and heptane. In chemical processing plants, natural gas liquids are typically cracked to produce more valuable chemicals such as olefins. Indeed, most of the liquid feedstock sent to cracking units (crackers or pyrolysis furnaces) is for generating petrochemicals feedstock. The natural gas liquid is typically a mixture of pentanes (n-C₅ around 50 wt. %, i-C₅ around 40 wt. %, and others)—understandably, there is variation in natural gas liquid composition based on the well from which it is recovered. The pyrolysis furnaces crack the components of natural gas liquid feedstock to produce high value olefins, such as ethylene, propylene, and butadiene, and aromatics such as benzene, etc. When lighter hydrocarbons such as ethylene are in high demand, enhancement in the yield of such lighter hydrocarbons at the outlet of crackers ultimately enhances overall profitability of the liquid cracking process. Because of widespread processing of natural gas liquids worldwide, there is a need to design integrated processes to meet the heightened demands with respect productivity and energy efficiency.

### BRIEF SUMMARY OF THE INVENTION

A method has been discovered that addresses the demands for increased productivity and energy efficiency with respect to the processing of natural gas liquids. The method involves converting an iso-alkane of natural gas liquid to normal-alkane via a reverse isomerization process. By implementing this reverse isomerization, the concentration of normal-alkanes in the feed to the cracking unit is increased as compared to when no reverse isomerization is carried out on the natural gas liquid. The increase in concentration of normal-alkanes when reverse isomerization is carried out results in an increase in production of light olefins and aromatics as compared to when no reverse isomerization is carried out. In other words, upgrading the natural gas liquid feedstock, before injecting it to crackers, can enhance light olefins yield from the natural gas liquid feedstock.

Accordingly, embodiments of the disclosure include a method of upgrading natural gas liquid feedstocks to enhance light olefins productivity from cracking. The method includes converting iso-alkane (e.g., iso-pentane) content of the natural gas liquid to a normal corresponding isomer of the iso-alkane (e.g., normal-pentane with respect to iso-pentane) by reverse-isomerization and thereby form a cracking unit feed having a higher concentration of normal isomers than the natural gas liquid. The method further includes processing the cracking unit feed to produce one or more light olefins and/or other value added hydrocarbons such as benzene and butadiene.

Embodiments of the disclosure include a method of producing light olefins and/or other value added hydrocarbons such as benzene and butadiene from natural gas liquid that comprises iso-alkane and normal-alkane. The method includes integration of converting some of the iso-alkane of the natural gas liquid to normal-alkane and thereby form a cracking unit feed having a higher concentration of normal-alkane than the original natural gas liquid. The method further includes processing the cracking unit feed to produce one or more light olefins and/or other value added hydrocarbons such as benzene and butadiene.

Embodiments of the disclosure include an integrated method of producing olefins and/or aromatics from natural gas liquid that mainly comprises iso-pentane and normal-pentane. The method includes hydrotreating the natural gas liquid to remove unwanted impurities and thereby produce a first stream. The method further involves separating the first stream in a separator to produce a second stream comprising primarily iso-pentane and isomerizing iso-pentane of the second stream in the presence of an active metal based catalyst to form normal-pentane. In this way, a third stream is produced that has a higher concentration of normal-pentane than the second stream. The method also includes removing methane and hydrogen from the third stream to form a fourth stream comprising normal-pentane and unconverted iso-pentane and separating the fourth stream in the same separator and/or using another separator, wherein the separating of the first stream and the separating of the fourth stream produces a cracking unit feed comprising primarily normal-pentane. The method then includes subjecting the one or more cracking unit feeds to conditions that crack molecules of the one or more cracking unit feeds to produce a product stream comprising mainly ethylene.

The following includes definitions of various terms and phrases used throughout this specification.

The terms “about” or “approximately” are defined as being close to as understood by one of ordinary skill in the art. In one non-limiting embodiment the terms are defined to be within 10%, preferably, within 5%, more preferably, within 1%, and most preferably, within 0.5%.

For the purposes of this disclosure, “X, Y, and/or Z” can be construed as X only, Y only, Z only, or any combination of two or more items X, Y, and Z (e.g., XYZ, XY, XZ, YZ).

The terms “wt. %”, “vol. %” or “mol. %” refer to a weight, volume, or molar percentage of a component, respectively, based on the total weight, the total volume, or the total moles of material that includes the component. In a non-limiting example, 10 moles of component in 100 moles of the material is 10 mol. % of component.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification, include any measurable decrease or complete inhibition to achieve a desired result.

The term “effective,” as that term is used in the specification and/or claims, means adequate to accomplish a desired, expected, or intended result.

The use of the words “a” or “an” when used in conjunction with the term “comprising,” “including,” “containing,” or “having” in the claims or the specification may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The process of the present disclosure can “comprise,” “consist essentially of,” or “consist of” particular ingredients, components, compositions, etc., disclosed throughout the specification.

The term “primarily,” as that term is used in the specification and/or claims, means greater than any of 50 wt. %, 50 mol. %, and 50 vol. %. For example, “primarily” may include 50.1 wt. % to 100 wt. % and all values and ranges there between, 50.1 mol. % to 100 mol. % and all values and ranges there between, or 50.1 vol. % to 100 vol. % and all values and ranges there between.

The term “light olefins,” refers to any olefin with a total of two to four carbon atoms.

The term “natural gas liquid,” refers to components of natural gas that have been separated from the gas state in the form of liquids, e.g., a liquid stream comprising mostly five to six carbon atoms.

Other objects, features and advantages of the present disclosure will become apparent from the following figures, detailed description, and examples. It should be understood, however, that the figures, detailed description, and examples, while indicating specific embodiments of the disclosure, are given by way of illustration only and are not meant to be limiting. Additionally, it is contemplated that changes and modifications within the spirit and scope of the disclosure will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

### BRIEF DESCRIPTION OF THE DRAWINGS

For a more complete understanding, reference is now made to the following descriptions taken in conjunction with the accompanying drawings, in which:

FIG. 1 shows an integrated system or scheme for processing natural gas liquid to produce one or more light olefins, according to embodiments of the disclosure;

FIG. 2 shows a generalized system for processing natural gas liquid to produce paraffinic rich stream using reverse isomerization, according to embodiments of the disclosure;

FIG. 3 shows a systematic method of processing natural gas liquid to produce a paraffinic rich stream using reverse isomerization according to embodiments of the disclosure;

FIG. 4 shows an integrated method of processing natural gas liquid to produce one or more light olefins according to embodiments of the disclosure; and

FIG. 5 demonstrates the effect of normal-pentane concentration change in natural gas liquid on ethylene productivity from cracking.

### DETAILED DESCRIPTION OF THE INVENTION

The present disclosure relates to an integrated process that involves introducing reverse isomerization into the cracking of natural gas liquid to produce products such as light olefins, benzene, and butadiene. According to embodiments of the disclosure, iso-pentane (i-C₅) of the natural gas liquid is converted to normal-pentane (n-C₅) before feeding to the cracking unit. This increase in the amount normal-pentane in the feed that is cracked to produce light olefins, benzene, and/or butadiene enhances the yield/productivity of light olefins (in particular ethylene). In other words, the pre-processing of natural gas liquid by reverse isomerization results in a n-C₅ rich feedstock to the cracking unit that enhances the overall productivity of light olefins.

### Integrated Systems for Processing Natural Gas Liquid

FIG. 1 shows an integrated system 10 for processing natural gas liquid to produce one or more light olefins, according to embodiments of the disclosure. According to embodiments of the disclosure, system 10 includes reverse isomerization unit 100. Reverse isomerization unit 100 has an inlet for receiving natural gas liquid 101 and is adapted to carry out reverse isomerization of iso-alkanes such as iso-pentane and thereby form a normal-alkane such as normal-pentane. In embodiments of the disclosure, reverse isomerization unit 100 has disposed therein a platinum based catalyst for catalyzing the reverse isomerization reaction. System 10, according to embodiments of the disclosure, further includes cracking unit 103, wherein an outlet of reverse isomerization unit 100 is in fluid communication with an inlet of cracking unit 103 such that cracking unit feed 102, which emanates from reverse isomerization unit 100, flows into cracking unit 103. Cracking unit 103 is configured for cracking hydrocarbons to produce valuable products such as light olefins, benzene, and butadiene. In embodiments of the disclosure, system 10 includes separation unit 105, which is adapted to receive product stream 104 from cracking unit 103 and to separate product stream 104 into components such as propylene, pygas, ethane and propane, methane, hydrogen, butadiene, C₄·s, and fuel oil.

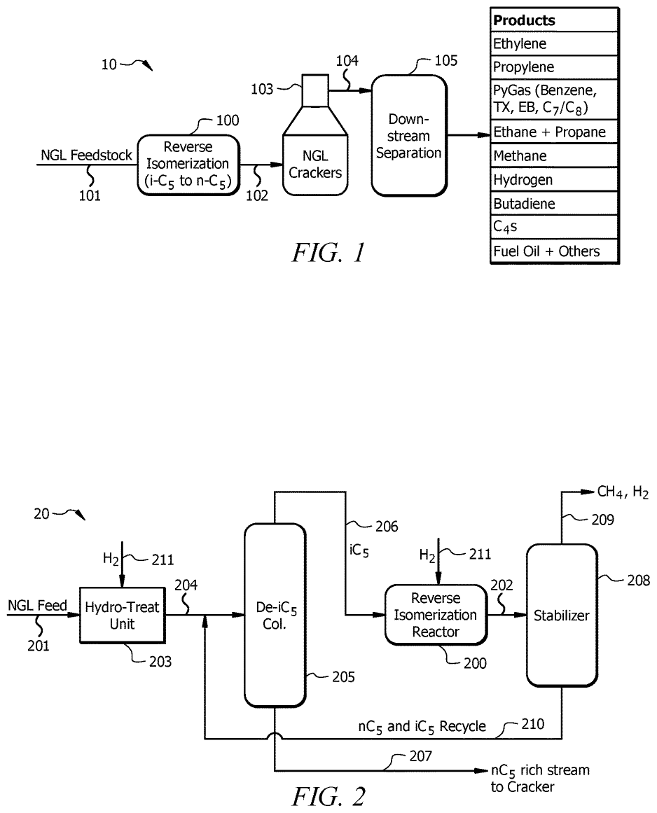

FIG. 2 shows system 20 for processing natural gas liquid to produce one or more light olefins, according to embodiments of the disclosure. System 20, in embodiments of the disclosure, includes various components for upgrading feedstock in the form of natural gas liquid 201 prior to sending the upgraded feedstock to a cracking unit for cracking. According to embodiments of the disclosure, system 20 includes hydro-treatment unit 203, which is adapted to receive and hydro-treat natural gas liquid 201 and thereby remove impurities such as sulfur compounds from natural gas liquid 201. In embodiments of the disclosure, hydrogen or hydrogen and chlorine are used to carry out the hydro-treating process. FIG. 2 shows hydrogen stream 211 being flowed through an inlet into hydro-treatment unit 203, in which the hydro-treatment of natural gas liquid 201 occurs.

System 20, according to embodiments of the disclosure, includes separation column 205, which is configured to separate hydro-treated NGL stream 204, which is an effluent from hydro-treatment unit 203. The system is not limited to the amount of columns shown in FIG. 2 and embodiments of the disclosure can include a plurality of separation columns. In embodiments of the disclosure, separation column 205 is a distillation column. Separation column 205, according to embodiments of the disclosure, is in fluid communication with hydro-treatment unit 203 and receives hydro-treated NGL stream 204 from hydro-treatment unit 203; after which separation column 205 separates hydro-treated NGL stream 204 into (1) iso-pentane stream 206, comprising primarily iso-pentane and (2) normal-pentane stream 207 comprising primarily normal-pentane.

In embodiments of the disclosure, system 20 further includes reverse isomerization unit 200, which is adapted to reverse isomerize iso-pentane in iso-pentane stream 206 to form normal-pentane. In this way, isomerized stream 202, emanating from reverse isomerization unit 200 has a higher concentration of normal-pentane than iso-pentane stream 206. In embodiments of the disclosure, reverse isomerization unit 200 has disposed therein a platinum based catalyst for catalyzing the reverse isomerization reaction. The system is not limited to the number of reverse isomerization reactors integrated, in parallel and/or series, and intermediate stages involved as shown in FIG. 2 as embodiments of the disclosure can include a plurality of isomerization reactors and other intermediate stages. According to embodiments of the disclosure, system 20 also includes stabilizer 208 for separating isomerized stream 202 into (1) lights 209 comprising primarily methane and hydrogen, collectively, and (2) recycle pentane stream 210 comprising primarily normal-pentane and iso-pentane collectively. In embodiments of the disclosure, stabilizer 208 is a distillation column. According to embodiments of the disclosure, an outlet of stabilizer 208 is in fluid communication with an inlet of separator column 205, so that recycle pentane stream 210 can be flowed from stabilizer 208 to separator column 205 for further processing-specifically, separation into iso-pentane and normal-pentane streams.

### Methods for Processing Natural Gas Liquids

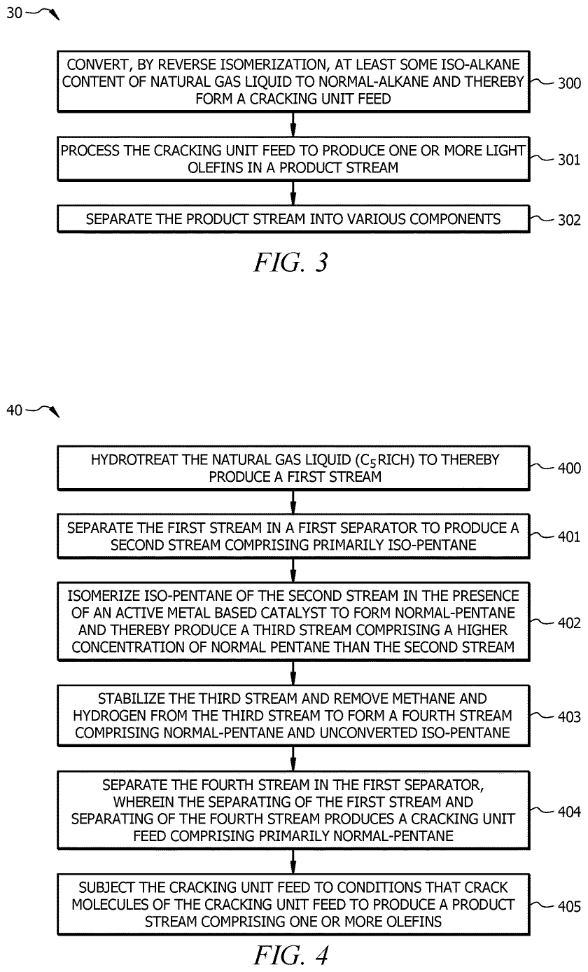

FIG. 3 shows method 30 for processing natural gas liquid to produce a paraffinic-rich stream according to embodiments of the disclosure. Method 30 may be implemented by system 10, in embodiments of the disclosure.

Method 30, according to embodiments of the disclosure, includes flowing natural gas liquid 101 into reverse isomerization unit 100 and converting, by reverse isomerization, at block 300, at least some iso-alkane content of natural gas liquid 101 to normal-alkane and thereby form cracking unit feed 102. According to embodiments of the disclosure, cracking unit feed 102 has a higher concentration of normal-alkane than natural gas liquid 101. In embodiments of the disclosure, the reverse isomerization is carried out, in reverse isomerization unit 100, under conditions including a temperature of 100 to 500° C. and a pressure of 1 to 50 bar, depending upon the catalyst nature and reaction requirements.

According to embodiments of the disclosure, the iso-alkane described above is iso-pentane, which is reversed isomerized to form normal-pentane, the normal-alkane described above. Natural gas liquid 101 can have varying compositions, based on its source, and in embodiments of the disclosure, natural gas liquid 101 comprises 20 to 80 wt. % iso-pentane and 20 to 80 wt. % normal-pentane.

Method 30, in embodiments of the disclosure, involves flowing cracking unit feed 102 from reverse isomerization unit 100 to cracking unit 103 and processing cracking unit feed 102, in cracking unit 103, to produce one or more light olefins as shown in block 301. In embodiments of the disclosure, the one or more olefins comprise ethylene. According to embodiments of the disclosure, product stream 104 includes ethylene as well as components such as propylene, benzene, ethane, propane, methane, hydrogen, butane, butene, pygas, and butadiene. Method 30, in embodiments of the disclosure, includes separation unit 105 separating product stream 104, at block 302, into various components such as ethylene as well as components such as propylene, pygas, ethane, propane, methane, hydrogen, butadiene, C₄s and fuel oil.

FIG. 4 shows method 40 for processing natural gas liquid to produce one or more light olefins according to embodiments of the disclosure. Method 40 may be implemented by system 20, in embodiments of the disclosure.

Method 40, in embodiments of the disclosure, includes receiving natural gas liquid 201 through an inlet into hydro-treatment unit 203. Hydro-treatment unit 203 is adapted to hydro-treat natural gas liquid 201. Natural gas liquid 201, in embodiments of the disclosure, comprises 20 to 80 wt. % iso-pentane and 20 to 80 wt. % normal-pentane. In embodiments of the disclosure, method 40 involves, at block 400, hydro-treatment unit 203 hydro-treating natural gas liquid 201 to remove impurities, such as sulfur compounds, from natural gas liquid 201. In embodiments of the disclosure, hydrogen (e.g., hydrogen stream 211) or hydrogen and chlorine are flowed into hydro-treatment unit 203 to carry out the hydro-treating process. The hydro-treating at block 400, according to embodiments of the disclosure, produces hydro-treated NGL stream 204.

According to embodiments of the disclosure, method 40 involves flowing hydro-treated NGL stream 204 from hydro-treatment unit 203 into separation column 205; and, at block 401, method 40 further involves separating hydro-treated NGL stream 204 (first stream) in separation column 205 (first separator) to produce iso-pentane stream 206 (second stream), which comprises primarily iso-pentane. Separation column 205, in embodiments of the disclosure is a distillation column. According to embodiments of the disclosure, iso-pentane stream 206 comprises 70 to 100 wt. % iso-pentane. Method 40 further includes flowing iso-pentane stream 206 to reverse isomerization unit 200, according to embodiments of the disclosure. And at block 402, method 40 involves, in embodiments of the disclosure, isomerizing iso-pentane of iso-pentane stream 206 in the presence of an active metal based catalyst (such as a platinum based catalyst) to form normal-pentane and thereby produce isomerized stream 202 (third stream), which comprises a higher concentration of normal-pentane than iso-pentane stream 206. The isomerizing at block 402, in embodiments of the disclosure is carried out under conditions within reverse isomerization unit 200 that includes a temperature of 80 to 480° C. and a pressure of 1 to 50 bar.

According to embodiments of the disclosure, method 40 involves flowing isomerized stream 202 to stabilizer 208 (second separator). The system is not limited to the number of stabilizers shown in FIG. 2 as embodiments of the disclosure may include a plurality of stabilizers. At block 403, method 40 includes removing methane and hydrogen from isomerized stream 202 to form recycle pentane stream 210 (fourth stream), which comprises normal-pentane and unconverted iso-pentane. In other words, stabilizer 208 stabilizes isomerized stream 202 and separates (1) lights 209 comprising primarily methane and hydrogen, collectively, and (2) recycle pentane stream 210 comprising primarily normal-pentane and iso-pentane, collectively.

Method 40, according to embodiments of the disclosure, includes flowing recycle pentane stream 210 to separation column 205 so that, at block 404, method 40 involves separating recycle pentane stream 210 in separation column 205; and in this way, the separating of hydro-treated NGL stream 204 and the separating of recycle pentane stream 210 produces normal-pentane stream 207 (cracking unit feed), which comprises primarily normal-pentane. Method 40 involves, at block 405, subjecting normal-pentane stream 207 to conditions that crack molecules of normal-pentane stream 207 to produce a product stream comprising one or more olefins. According to embodiments of the disclosure, the product stream comprises one or more of: ethylene, propylene, benzene, ethane, propane, methane, hydrogen, and butadiene.

The typical products stream from NGL cracking is shown below in Table 1:

TABLE 1

| Products | Wt. % |
|---|---|
| Ethylene | 38.8 |
| Propylene | 14.2 |
| Pygas (Benzene, TX, EB, C7/C8, Raf) | 9.1 |
| Ethane + Propane | 2.2 |
| Methane/Fuel Gas | 17.6 |
| Hydrogen | 1.5 |
| Butadiene | 5.1 |
| C4s | 8.5 |
| Fuel Oil | 2.9 |

Method 40, in embodiments of the disclosure, could process recycle pentane stream 210 differently than described in block 404 and block 405, specifically, wherein the separating of hydro-treated NGL stream 204 in separator column 205 produces a first cracking unit feed and method 40 further includes separating recycle pentane stream 210 in a second separator (different from separator column 205) to produce a second cracking unit feed comprising primarily normal-pentane. According to embodiments of the disclosure, method 40 involves subjecting the first cracking unit feed and the second cracking unit feed to conditions that cracks molecules of the first cracking unit feed and the second cracking unit feed to produce at least some of a product stream comprising one or more light olefins. According to embodiments of the disclosure, the product stream comprises one or more of: ethylene, propylene, benzene, ethane, propane, methane, hydrogen, and butadiene.

Although embodiments of the present disclosure have been described with reference to blocks of FIG. 3 and FIG. 4, it should be appreciated that operation of the present disclosure is not limited to the particular blocks and/or the particular order of the blocks illustrated in FIG. 3 and FIG. 4. Accordingly, embodiments of the disclosure may provide functionality as described herein using various blocks in a sequence different than that of FIG. 3 and FIG. 4.

The systems and processes described herein can also include various equipment that is not shown and is known to one of skill in the art of chemical processing. For example, some controllers, piping, computers, valves, pumps, heaters, thermocouples, pressure indicators, mixers, heat exchangers, and the like may not be shown.

As part of the disclosure of the present invention, specific examples are included below. The examples are for illustrative purposes only and are not intended to limit the invention. Those of ordinary skill in the art will readily recognize parameters that can be changed or modified to yield essentially the same results.

### EXAMPLE

### Simulation to Determine the Effect of Normal-Pentane Concentration in NGL Feedstock to a Cracking Unit

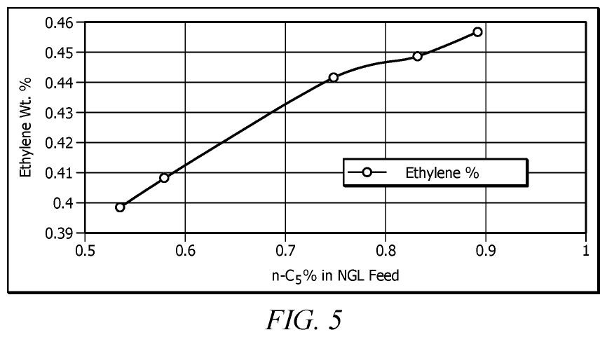

Extensive simulation based estimations were conducted using various simulation platforms, with a liquid furnace design. Under optimal operational conditions of liquid furnaces for ethylene maximization, such as high severity coils, P/E 0.4, etc. were used for the simulations. FIG. 5 shows the comparative effect of n-C₅ concentration change in natural gas liquid (NGL) on ethylene productivity, under identical operational conditions.

In the context of the present invention, at least the following 21 embodiments are disclosed. Embodiment 1 is an integrated method of processing natural gas liquid to produce one or more light olefins, further used as a feedstock in petrochemical production. The method includes converting, by reverse isomerization, at least some iso-alkane content of the natural gas liquid to normal-alkane and thereby form a cracking unit feed having a higher concentration of normal-alkane than the natural gas liquid. The method further includes processing the cracking unit feed to produce one or more light olefins. Embodiment 2 is the method of embodiment 1, wherein the reverse isomerization is carried out under conditions including a temperature of 100 to 500° C. and a pressure of 1 to 50 bar. Embodiment 3 is the method of any of embodiments 1 and 2, wherein the iso-alkane is iso-pentane and the normal-alkane is normal-pentane. Embodiment 4 is the method of any of embodiments 1 to 3, wherein the natural gas liquid contains 20 to 80 wt. % iso-pentane and 20 to 80 wt. % normal-pentane. Embodiment 5 is the method of any of embodiments 1 to 4, wherein the one or more light olefins contain ethylene. Embodiment 6 is the method of any of embodiments 1 to 5, wherein the processing of the cracking unit feed produces a product stream containing a selection from the list consisting of: ethylene, propylene, benzene, ethane, propane, methane, hydrogen, butane, butene, pygas, and butadiene. Embodiment 7 is the method of any of embodiments 1 to 6, wherein the product stream contains primarily ethylene.

Embodiment 8 is a method of processing natural gas liquid containing iso-pentane and normal-pentane. The method includes hydrotreating the natural gas liquid to produce a first stream. The method further includes separating the first stream in a first separator to produce a second stream containing primarily iso-pentane. The method still further includes isomerizing iso-pentane of the second stream in the presence of an active metal based catalyst to form normal-pentane and thereby produce a third stream containing a higher concentration of normal-pentane than the second stream. The method yet further includes removing methane and hydrogen from the third stream to form a fourth stream containing normal-pentane and unconverted iso-pentane. Embodiment 9 is the method of any of embodiments 1 to 8, further including separating the fourth stream in the first separator, wherein the separating of the first stream and the separating of the fourth stream produces a cracking unit feed containing primarily normal-pentane. The method also includes subjecting the cracking unit feed to conditions that crack molecules of the cracking unit feed to produce a product stream containing one or more olefins. Embodiment 10 is the method of any of embodiments 1 to 9, wherein the one or more olefins contains ethylene. Embodiment 11 is the method of any of embodiments 1 to 10, wherein the subjecting of the cracking unit feed to the conditions produces a product stream containing a selection from the list consisting of: ethylene, propylene, benzene, ethane, propane, methane, hydrogen, and butadiene. Embodiment 12 is the method of any of claims 1 to 11, wherein the first separator is a distillation column. Embodiment 13 is the method of any of embodiments 1 to 12, wherein the separating of the first stream in the first separator produces a first cracking unit feed. The method further includes separating the fourth stream in a second separator to produce a second cracking unit feed containing primarily normal-pentane. The method still further includes subjecting the first cracking unit feed and the second cracking unit feed to conditions that crack molecules of the first cracking unit feed and the second cracking unit feed to produce at least some of a product stream containing one or more light olefins. Embodiment 14 is the method of any of embodiments 1 to 13, wherein the one or more light olefins contain ethylene. Embodiment 15 is the method of any of embodiments 1 to 14, wherein the subjecting of the first cracking unit feed and the second cracking unit feed to the conditions results in the product stream containing a selection from the list consisting of: ethylene, propylene, benzene, ethane, propane, methane, hydrogen, and butadiene. Embodiment 16 is the method of any of embodiments 1 to 15, wherein the first separator and the second separator are distillation columns. Embodiment 17 is the method of any of embodiments 1 to 16, wherein the isomerizing is carried out under conditions including a temperature of 100 to 500° C. and a pressure of 1 to 50 bar. Embodiment 18 is the method of any of embodiments 1 to 17, wherein the second stream includes 50 to 100 wt. % iso-pentane.

Embodiment 19 is the method of any of embodiments 1 to 18, wherein the natural gas liquid includes 20 to 80 wt. % iso-pentane, 20 to 80 wt. % normal-pentane. Embodiment 20 is the method of any of embodiments 1 to 19, wherein the reverse isomerization will be a catalytic process. Embodiment 21 is the method of any of embodiments 1 to 20, wherein the active metal based catalyst may comprise platinum.

All embodiments described above and herein can be combined in any manner unless expressly excluded.

Although embodiments of the present application and their advantages have been described in detail, it should be understood that various changes, substitutions and alterations can be made herein without departing from the spirit and scope of the embodiments as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the above disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

What is claimed is:

1. A method of processing natural gas liquid to produce one or more light olefins, the method comprising:
converting, by reverse isomerization, at least some iso-alkane content of the natural gas liquid to normal-alkane and thereby form a cracking unit feed having a higher concentration of normal-alkane than the natural gas liquid; and
processing the cracking unit feed to produce one or more light olefins.

2. The method of claim 1, wherein the reverse isomerization is carried out under conditions including a temperature of 100 to 500° C. and a pressure of 1 to 50 bar to maximize n-pentane as feed to the cracking unit.

3. The method of claim 1, wherein the iso-alkane is iso-pentane and the normal-alkane is normal-pentane.

4. The method of claim 1, wherein the natural gas liquid comprises 20 to 80 wt. % iso-pentane and 20 to 80 wt. % normal-pentane.

5. The method of claim 1, wherein the processing of the natural gas liquid produces one or more light olefins that comprise ethylene.

6. The method of claim 1, wherein the processing of the cracking unit feed produces a product stream comprising one or more of: ethylene, propylene, benzene, ethane, propane, methane, hydrogen, butane, butene, pygas, and butadiene.

7. The method of claim 6, wherein the product stream comprises primarily ethylene and propylene.

8. A method of processing natural gas liquid comprising iso-pentane and normal-pentane, the method comprising:
hydrotreating the natural gas liquid to produce a first stream;
separating the first stream in a first separator to produce a second stream comprising primarily iso-pentane;
isomerizing iso-pentane of the second stream in the presence of an active metal based catalyst to form normal-pentane and thereby produce a third stream comprising a higher concentration of normal-pentane than the second stream;
removing methane and hydrogen from the third stream to form a fourth stream comprising normal-pentane and unconverted iso-pentane.

9. The method of claim 8, further comprising:
separating the fourth stream in the first separator, wherein the separating of the first stream and the separating of the fourth stream produces a cracking unit feed comprising primarily normal-pentane; and
subjecting the cracking unit feed to conditions that crack molecules of the cracking unit feed to produce a product stream comprising one or more olefins.

10. The method of claim 9, wherein the one or more olefins comprise ethylene.

11. The method of claim 9, wherein the subjecting of the cracking unit feed to the conditions produces a product stream comprising a selection from the list consisting of: ethylene, propylene, benzene, ethane, propane, methane, hydrogen, butane, butene, pygas, and butadiene.

12. The method of claim 9, wherein the first separator is a distillation column.

13. The method of claim 8, wherein the separating of the first stream in the first separator produces a first cracking unit feed, comprising primarily normal alkane, and the method further comprising: separating the fourth stream in a second separator to produce a second cracking unit feed comprising primarily normal-pentane; and
subjecting the first cracking unit feed and the second cracking unit feed to conditions that crack molecules of the first cracking unit feed and the second cracking unit feed to produce a product stream comprising one or more light olefins.

14. The method of claim 13, wherein the one or more light olefins comprise ethylene.

15. The method of claim 13, wherein the subjecting of the first cracking unit feed and the second cracking unit feed to the conditions results in the product stream comprising a selection from the list consisting of, but not limited to: ethylene, propylene, benzene, ethane, propane, methane, hydrogen, butane, butene, pygas, and butadiene.

16. The method of claim 13, wherein the first separator and the second separator are distillation columns.

17. The method of claim 8, wherein the isomerizing is carried out under conditions including a temperature of 100 to 500° C. and a pressure of 1 to 50 bar.

18. The method of claim 8, wherein the second stream comprises 60 to 100 wt. % iso-pentane.

19. The method of claim 8, wherein the natural gas liquid comprises 20 to 80 wt. % iso-pentane, 20 to 80 wt. % normal-pentane.

20. The method of claim 8, wherein the reverse isomerization is carried out using active metal based catalyst.
